(19) United States
(12) Patent Application Publication
(10) Pub. No.: US 2025/0152705 A1
(43) Pub. Date: May 15, 2025

(54) COMBINATION THERAPY COMPRISING AN ANTI-IL-23 ANTIBODY AND A CORTICOSTEROID FOR TREATING PSORIASIS

(71) Applicant: SUN PHARMACEUTICAL INDUSTRIES LIMITED, Mumbai, Maharashtra (IN)

(72) Inventors: Jerry BAGEL, East Windsor, NJ (US); Kristin NOVAK, East Windsor, NJ (US); Elise NELSON, East Windsor, NJ (US)

(21) Appl. No.: 18/728,567

(22) PCT Filed: Jan. 12, 2023
(86) PCT No.: PCT/IB2023/050294

§ 371 Date: Jul. 12, 2024

Related U.S. Application Data
(60) Provisional application No. 63/299,181, filed on Jan. 13, 2022.

(51) Int. Cl. A61K 39/395; A61K 9/00; A61K 9/06; A61K 31/58; A61K 39/00; A61P 17/06

(52) U.S. Cl. CPC A61K 39/3955; A61K 9/0014; A61K 9/0019; A61K 9/06; A61K 31/58; A61P 17/06; A61K 2039/54; A61K 2039/545

(57) ABSTRACT

The present disclosure relates to a combination of the anti-IL-23 antibody tildrakizumab in combination with a corticosteroid for use in the treatment of plaque psoriasis.

### CROSS-REFERENCE TO RELATED APPLICATION(S)

The present application claims priority to U.S. Provisional Application 63/299,181 filed on Jan. 13, 2022, the entire contents of which are incorporated herein by reference.

### FIELD OF THE INVENTION

The present invention provides combination therapy comprising IL-23 antagonist and corticosteroid. The combination therapy of the present invention is useful for the treatment, prevention or alleviation of plaque psoriasis. The addition of corticosteroid to IL-23 inhibitors therapy potentiates the IL-23 inhibitor's anti-psoriatic therapeutic effect.

### BACKGROUND OF THE INVENTION

Psoriasis is a chronic inflammatory skin disease prevalent in approximately 3% of adults in the US (JAMA Dermatol. 2021; 157(8):940-946). It is characterized by marked inflammation and increased epidermal thickness resulting from infiltration of the skin with activated T cells and abnormal proliferation and differentiation of keratinocytes. Moderate to severe psoriasis is associated with a number of comorbidities including metabolic syndrome, cardiovascular and cerebrovascular diseases, depression, and anxiety (Acad Dermatol. 2017; 76(3):377-390). Patients may also develop mental health conditions and have reduced quality of life (Patient Relat Outcome Meas. 2015; 6167-177).

Therapeutic options include topical therapy for limited psoriasis, and phototherapy, systemic medications and biologic agents for extensive psoriasis (J Am Acad Dermatol. 2008; 58(5):826-850). For plaque psoriasis, the National Psoriasis Foundation suggests an acceptable treatment response of ≤3% affected body surface area (BSA) and a target response of BSA≤1% after treating for 3 months (J Am Acad Dermatol. 2017; 76(2):290-298).

Drugs that inhibit TNF-a have been approved for the treatment of psoriasis, including the biologics infliximab (REMICADE®), adalimumab (HUMIRA®), and etanercept (ENBREL®). Inflixmab and adalimumab are, respectively, a chimeric antibody and a fully human antibody that bind to human TNF-a. Etanercept is a fusion protein consisting of the extracellular region of the human p75 TNF receptor fused to an Fe portion of an antibody, which is currently in use as a systemic treatment for moderate to severe plaque psoriasis.

Among biologic agents, tildrakizumab is a humanized IgG1 monoclonal antibody that selectively binds to interleukin-23 (IL-23) and inhibits its receptor interaction. Randomized, controlled trials showed that tildrakizumab monotherapy was efficacious compared with placebo for psoriasis treatment and well tolerated in patients with chronic moderate to severe plaque psoriasis. However, in a cohort study, almost half of patients did not achieve BSA 3% with tildrakizumab monotherapy at week 16.

Surprisingly, the inventors of the present invention have found that 4 weeks of adjunctive use of topical halcinonide ointment effectively improved all outcomes in these patients, reflected in the decreased mean BSA, PGA and BSA×PGA and the increased proportion of all patients with BSA≤3% from week 16 to week 20. Moreover, the enhanced responses are maintained through week 24 after the additional halcinonide ointment has been stopped for 4 weeks. Further, the inventors have found that the patient response to tildrakizumab could be augmented with the addition of halcinonide ointment, without the need to increase tildrakizumab doses or switch to a new biologic agent.

### SUMMARY OF THE INVENTION

Accordingly, the present invention provides combination therapy comprising an IL-23 antagonist and a corticosteroid. The combination therapy of the present invention is useful for the treatment, prevention or alleviation of plaque psoriasis. The addition of a corticosteroid to IL-23 inhibitors therapy potentiates the IL-23 inhibitor's anti-psoriatic therapeutic effect.

Therefore, the present invention provides one or more of the following embodiments.

In one embodiment, the present invention provides a method for treating a patient suffering from moderate to severe plaque psoriasis, which method comprises:
selecting a patient who has
a PASI score of ≥12
≥10% body surface area (BSA) affected by psoriasis and/or
a PGA score of ≥3
administering an IL-23 inhibitor to said patient at a dose of about 100 milligrams by subcutaneous injection at weeks 0, 4 and 16 week;
at week 16, applying a corticosteroid twice daily to the patients with BSA >3% for 4 weeks and then continued without the corticosteroid for an additional four weeks follow up, and
changes from baseline in BSA, PGA, PGA×BSA, PSAI and DLQI are noted; wherein the patients receiving the corticosteroid achieve BSA≤1% to ≤3% at week 20 and maintain the response level through week 24.

In another embodiment, the present invention provides a method for treating a patient suffering from moderate to severe plaque psoriasis wherein the percentage of reduction from baseline in mean BSA, PGA, and PGA×BSA significantly increases at week 20 after 4 weeks of adjunctive corticosteroid therapy and the response is maintained at week 24 after corticosteroid therapy stops for 4 weeks.

In yet another embodiment, the present invention provides a method for treating a patient suffering from moderate to severe plaque psoriasis wherein the percentage of reduction in PSAI (75, 90 and 100) from baseline significantly increases at week 20 after 4 weeks of adjunctive corticosteroid therapy and the response is maintained at week 24 after corticosteroid therapy stops for 4 weeks.

In still yet another embodiment, the present invention provides a method for treating a patient suffering from moderate to severe plaque psoriasis wherein the reduction in DLQI from baseline significantly increases at week 20 after 4 weeks of adjunctive corticosteroid therapy and the response is maintained at week 24 after corticosteroid therapy stops for 4 weeks.

In still yet another embodiment, the present invention provides a method for treating a patient suffering from moderate to severe plaque psoriasis wherein the corticosteroid is administered as a topical preparation.

In one embodiment, the present invention provides a method for treating a patient suffering from moderate to severe plaque psoriasis wherein the topical preparation is in the form of an ointment, a powder, a gel ointment, an emollient, a cream, an adhesive patch or strip, a shampoo, a spray, foam, a gel, a liquid or a solution.

In another embodiment, the present invention provides a method for treating a patient suffering from moderate to severe plaque psoriasis wherein the corticosteroid is administered as an ointment, preferably a 0.1% ointment.

In yet another embodiment, the present invention provides a method for treating a patient suffering from moderate to severe plaque psoriasis wherein the corticosteroid selected from beclomethasone dipropionate, betamethasone valerate, budesonide, clobetasol propionate, clobetasone butyrate, clobetasol propionate, clocortolone acetate, clocortolone pivalate, corticosterone, cortisone, esoximetasone acetatefluocinolone acetonide, halcinonide, halobetasol propionate, hydrocortisone acetate, hydrocortisone butyrate, hydrocortisone probutate, hydrocortisone phos-phate, hydrocortisone succinate sodium, methylprednisolone acetate, prednisolone or prednisone. In one embodiment, the corticosteroid is halcinonide. In another embodiment, the corticosteroid is prednisone.

In still yet another embodiment, the present invention provides a method for treating a patient suffering from moderate to severe plaque psoriasis wherein the IL-23 inhibitor is selected from guselkumab, risankizumab or tildrakizumab.

In another embodiment, the present invention provides a method for treating a patient suffering from moderate to severe plaque psoriasis wherein the IL-23 antagonist is tildrakizumab.

In yet another embodiment, the present invention provides a method for treating a psoriasis patient who is receiving an IL-23 antagonist comprising continuing treatment with the IL-23 antagonist, initiating treatment with a topical preparation containing a corticosteroid, continuing treatment with the topical preparation containing the corticosteroid for not more than 4 weeks and then discontinuing treatment with the topical preparation, wherein the patient percentage achieving BSA ≤3% is increased after just 4 weeks of additional corticosteroid applied to those who did not achieve an adequate response to IL-23 antagonist alone, and the said improvements are maintained 4 weeks after corticosteroid treatment is stopped.

In yet another embodiment, the present invention provides a method for treating a psoriasis patient who is receiving an IL-23 antagonist wherein the percentage of reduction from baseline in mean BSA, PGA, and PGA×BSA significantly increases at week 20 after 4 weeks of adjunctive corticosteroid therapy; the percentage of reduction in PSAI (75, 90 and 100) from baseline significantly increases at week 20 after 4 weeks of adjunctive corticosteroid therapy; the reduction in DLQI from baseline significantly increases at week 20 after 4 weeks of adjunctive corticosteroid therapy; and the response (all outcomes) is maintained at week 24 after corticostreoid stops for 4 weeks.

In yet another embodiment, the present invention provides a method for treating a psoriasis patient who is receiving an IL-23 antagonist wherein the patient continues the treatment with the IL-23 antagonist for at least about 16 weeks.

In yet another embodiment, the present invention provides a method for treating a psoriasis patient who is receiving an IL-23 antagonist wherein the IL-23 antagonist is guselkumab, risankizumab or tildrakizumab.

In yet another embodiment, the present invention provides a method for treating a psoriasis patient who is receiving an IL-23 antagonist wherein the IL-23 antagonist is tildrakizumab

In still yet another embodiment, the present invention provides a method for treating a psoriasis patient who is receiving an IL-23 antagonist wherein corticosteroid is administered as topical preparation.

In one embodiment, the present invention provides a method for treating a psoriasis patient who is receiving an IL-23 antagonist wherein the topical preparation is in the form of an ointment, a powder, a gel ointment, an emollient, a cream, an adhesive patch or strip, a shampoo, a spray, foam, a gel, a liquid or a solution.

In yet another embodiment, the present invention provides a method for treating a psoriasis patient who is receiving an IL-23 antagonist wherein the corticosteroid is selected from beclomethasone dipropionate, betamethasone valerate, budesonide, clobetasol propionate, clobetasone butyrate, clobetasol propionate, clocortolone acetate, clocortolone pivalate, corticosterone, cortisone, esoximetasone acetatefluocinolone acetonide, halcinonide, halobetasol propionate, hydrocortisone acetate, hydrocortisone butyrate, hydrocortisone probutate, hydrocortisone phos-phate, hydrocortisone succinate sodium, methylprednisolone acetate, prednisolone, prednisone or halcinonide, preferably halcinodine.

In still yet another embodiment, the present invention provides a method for treating a psoriasis patient who is receiving an IL-23 antagonist wherein corticosteroid is a halcinonide ointment, preferably a 0.1% ointment.

### BRIEF DESCRIPTION OF THE DRAWING

FIG. 1 Proportions of patients who achieved BSA≤3% and BSA≤1% at weeks 16, 20, and 24 (A) in all patients and (B) in patients with BSA >3% who received halcinonide at week 16.

FIG. 2 Mean BSA involvement at different time points for all patients, patients with BSA≤3% at week 16, and patients with BSA >3% at week 16. (n indicates number of patients with data available for each study visit).

FIG. 3 Mean PGA score different time points for all patients, patients with BSA≤3% at week 16, and patients with BSA >3% at week 16. (n indicates number of patients with data available for each study visit).

FIG. 4 Mean PGA×BSA at different time points for all patients, patients with BSA≤3% at week 16, and patients with BSA >3% at week 16. (n indicates number of patients with data available for each study visit).

FIG. 5 Proportions of patients achieving PASI 75, PASI 90, and PASI 100 responses in all patients at weeks 16, 20, and 24.

FIG. 6 Improvement in quality of life. (A) Proportions of patients achieving DLQI of 0 or 1 and (B) Mean DLQI at different time points for all patients, patients with BSA≤3% at week 16, and patients with BSA >3% at week 16. (n indicates number of patients with data available for each study visit).

The aforementioned aspects and embodiments, and other aspects, objects, features and advantages of the present invention will be apparent from the following detailed description.

### DETAILED DESCRIPTION OF THE INVENTION

In a cohort study, almost half of patients did not achieve BSA 3% with tildrakizumab monotherapy at week 16. Thus, there is a need for developing a new therapy involving adjunctive therapy or switching to a new therapy. In the present invention, the proportion of all patients achieving BSA≤3% and PASI 75 is increased after just 4 weeks of additional halcinonide ointment applied to those who do not achieve an adequate response to tildrakizumab alone. All disease activity outcomes and patient quality of life are improved with tildrakizumab monotherapy and further with the addition of halcinonide ointment. Importantly, the improvements are maintained 4 weeks after halcinonide ointment is stopped. In addition, tildrakizumab alone or in combination with halcinonide ointment is well tolerated and no SAEs are noted.

Accordingly, there is provided a method for treating a patient suffering from moderate to severe plaque psoriasis, which method comprises a) selecting a patient who has a PASI score of ≥12; ≥10% body surface area (BSA) affected by psoriasis and/or a PGA score of ≥3; b) administering an IL-23 antagonist, preferably tildrakizumab, to said patient at a dose of about 100 milligrams by subcutaneous injection at weeks 0, 4 and 16 week; c) at week 16, applying halcinonide twice daily to the patients with BSA >3% for 4 weeks and then continued without halcinonide for an additional four weeks follow up, and changes from baseline in BSA, PGA, PGA×BSA, PSAI and DLQI are noted; wherein:
a. the patients receiving halcinonide achieve BSA≤1% to ≤3% at week 20;
b. the percentage of reduction from baseline in mean BSA, PGA, and PGA×BSA significantly increases at week 20 after 4 weeks of adjunctive halcinonide therapy;
c. the percentage of reduction in PSAI (75, 90 and 100) from baseline significantly increases at week 20 after 4 weeks of adjunctive halcinonide therapy;
d. the reduction in DLQI from baseline significantly increases at week 20 after 4 weeks of adjunctive halcinonide therapy; and
e. the response is maintained at week 24 after halcinonide ointment stops for 4 weeks.

In another embodiment, there is provided a method for treating a psoriasis patient who is receiving IL-23 antagonist comprising continuing treatment with IL-23 antagonist, initiating treatment with a topical preparation containing a corticosteroid, continuing treatment with the topical preparation containing the corticosteroid for not more than 4 weeks and then discontinuing treatment with the topical preparation, wherein the patient percentage achieving BSA≤3% and PASI (75, 90 or 100) increased after just 4 weeks of additional corticosteroid applied to those who did not achieve an adequate response to IL-23 antagonist alone, and the said improvements are maintained 4 weeks after corticosteroid treatment is stopped.

Next, as used herein the following definitions apply unless clearly indicated otherwise. For example, the term “IL-23 antagonist or inhibitor” target a type of cytokine called IL-23, which plays a role in a signaling pathway that triggers inflammation. IL-23 inhibitors block the action of IL-23, which can help limit the inflammation that causes psoriasis symptoms. IL-23 inhibitors may help reduce the amount of skin that psoriasis affects and also relieve symptoms, such as itching, pain, and skin tightness. FDA has approved three IL-23 inhibitors for the treatment of moderate-to-severe psoriasis in adults. These are Guselkumab (TREMFYA), Risankizumab-rzaa (SKYRIZI) and Tildrakizumab-asmn (ILUMYA).

Guselkumab, an interleukin-23 blocker, is a human immunoglobulin G1 lambda (IgG1λ) monoclonal antibody. Guselkumab is produced in a mammalian cell line using recombinant DNA technology. TREMFYA Injection is a sterile, preservative free, clear, colorless to light yellow solution that may contain small translucent particles. Each single-dose prefilled syringe for subcutaneous use contains 100 mg of guselkumab in 1 mL. TREMFYA is supplied as a single dose solution in a 1 mL glass syringe with a 27G, half inch fixed needle assembled in a passive needle guard delivery system. The standard dosage is one injection at the start of treatment, one injection in the fourth week of treatment, and one injection every 8 weeks afterward. Each TREMFYA prefilled syringe delivers 1 mL of solution containing guselkumab (100 mg), Lhistidine (0.6 mg), L-histidine monohydrochloride monohydrate (1.5 mg), polysorbate 80 (0.5 mg), sucrose (79 mg) and water for injection at pH 5.8.

In another embodiment, there is provided a method for treating a psoriasis patient who is receiving Guselkumab comprising continuing treatment with Guselkumab, initiating treatment with a topical preparation containing a corticosteroid, continuing treatment with the topical preparation containing the corticosteroid for not more than 4 weeks and then discontinuing treatment with the topical preparation, wherein the patient percentage achieving BSA≤3% and PASI (75, 90 or 100) increased after just 4 weeks of additional corticosteroid applied to those who did not achieve an adequate response to Guselkumab alone, and the said improvements are maintained 4 weeks after corticosteroid treatment is stopped.

Risankizumab-rzaa, an interleukin-23 antagonist, is a humanized immunoglobulin G1 (IgG1) monoclonal antibody. Risankizumab-rzaa is produced in a mammalian cell line using recombinant DNA technology. SKYRIZI (risankizumab-rzaa) injection is a sterile, preservative-free, colorless to slightly yellow, and clear to slightly opalescent solution for subcutaneous use. Each prefilled syringe delivers 0.83 mL containing 75 mg risankizumab-rzaa, disodium succinate hexahydrate (0.88 mg), polysorbate 20 (0.17 mg), sorbitol (34 mg), succinic acid (0.049 mg), and Water for Injection, USP.

In yet another embodiment, there is provided a method for treating a psoriasis patient who is receiving Risankizumab comprising continuing treatment with Risankizumab, initiating treatment with a topical preparation containing a corticosteroid, continuing treatment with the topical preparation containing the corticosteroid for not more than 4 weeks and then discontinuing treatment with the topical preparation, wherein the patient percentage achieving BSA≤3% and PASI (75, 90 or 100) increased after just 4 weeks of additional corticosteroid applied to those who did not achieve an adequate response to Risankizumab alone, and the said improvements are maintained 4 weeks after corticosteroid treatment is stopped.

Tildrakizumab-asmn, a humanized IgG1/k antibody, specifically binds to the p19 subunit of interleukin-23 (IL-23). Tildrakizumab-asmn is produced in a recombinant Chinese hamster ovary (CHO) cell line and has an approximate molecular mass of 147 kilodaltons. ILUMYA (tildrakizumab-asmn) injection, for subcutaneous use, is a sterile, clear to slightly opalescent, colorless to slightly yellow solution. ILUMYA is supplied in a single-dose prefilled syringe with a glass barrel and 29-gauge fixed, ½-inch needle. The syringe is fitted with a passive needle guard and a needle cover. Each 1 mL single-dose prefilled syringe contains 100 mg of tildrakizumab-asmn formulated in: Lhistidine (0.495 mg), L-histidine hydrochloride monohydrate (1.42 mg), polysorbate 80 (0.5 mg), sucrose (70.0 mg), and Water for Injection, USP with a pH of 5.7-6.3.

In a particular embodiment, the present invention provides a method for treating a psoriasis patient who is receiving tildrakizumab comprising continuing treatment with tildrakizumab, initiating treatment with a topical preparation containing a corticosteroid, continuing treatment with the topical preparation containing the corticosteroid for not more than 4 weeks and then discontinuing treatment with the topical preparation, wherein the patient percentage achieving BSA≤3% and PASI (75, 90 or 100) increased after just 4 weeks of additional corticosteroid applied to those who did not achieve an adequate response to tildrakizumab alone, and the said improvements are maintained 4 weeks after corticosteroid treatment is stopped.

The term “corticosteroid” refers to a group of hormones secreted by the adrenal cortex and analogues thereof. “Corticosteroid” encompasses glucocorti-coids, mineralocorticoids, such as aldosterone, and sex steroids. The glucocorticoids, as typified by cortisol, bind to glucocorticoid receptors and have anti-inflammatory, metabolic, and immunoregulatory effects and regulate key homeostatic functions. Some corticosteroids, such as, for example, cortisone, have both glucocorticoid and mineralocorticoid effects, while others, have primarily glucocorticoid effects.

“Topical preparations containing corticosteroid” can be in the form of an ointment, a powder, a gel ointment, an emollient, a cream, an adhesive patch or strip, a shampoo, a spray, foam, a gel, or a liquid.

Examples of corticostreoid include, but not limited to beclomethasone dipropionate, betamethasone valerate, budesonide, clobetasol propionate, clobetasone butyrate, clobetasol propionate, clocortolone acetate, clocortolone pivalate, corticosterone, cortisone, esoximetasone acetatefluocinolone acetonide, halcinonide, halobetasol hydrocortisone propionate, acetate, hydrocortisone butyrate, hydrocortisone probutate, hydrocortisone phosphate, hydrocortisone succinate sodium, methylprednisolone acetate (or disodium phosphate, sodium succinate, or aceponate), prednisolone, halcinonide, prednisone, or the like.

The term “halcinonide” as used herein refers HALOG chemically designated as 21-chloro-9-fluoro-11β,16α,17-trihydroxypregn-4-ene-3,20-dione cyclic 16,17-acetal with acetone. Each gram of 0.1% HALOG (Halcinonide Cream, USP) contains 1 mg halcinonide in a specially formulated cream base consisting of cetyl alcohol, dimethicone 350, glyceryl monostearate NF XII, isopropyl palmitate, polysorbate 60, propylene glycol, purified water, and titanium dioxide.

Each mL of 0.1% HALOG solution (Halcinonide Topical Solution, USP) contains 1 mg halcinonide, edetate disodium, polyethylene glycol 300, and purified water with butylated hydroxytoluene as an antioxidant.

Each gram of 0.1% HALOG ointment (Halcinonide Ointment, USP) contains 1 mg halcinonide in Plastibase® (Plasticized Hydrocarbon Gel), a mineral oil and polyethylene gel base, polyethylene glycol 300, polyethylene glycol 400, polyethylene glycol 1450, and polyethylene glycol 6000 distearate with butylated hydroxytoluene as an antioxidant.

As used herein, the term “about”, refers to any value which lies within the range defined by a variation of up to ±15% of the value.

The present invention is illustrated below by reference to the following example. However, one skilled in the art will appreciate that specific methods and results discussed are merely illustrative of the invention, as innumerable variations, modifications, applications, and extensions of these embodiments and principles can be made without departing from the spirit and scope of the invention.

### EXAMPLE 1

A prospective, open-label study which evaluated the effectiveness and safety of tildrakizumab plus topical halcinonide ointment in psoriasis patients

### Methods

### Study Design and Participants

This was a single center, prospective, open-label study to evaluate the effectiveness and safety of tildrakizumab (ILUMYA® [tildrakizumab-asmn] in combination with halcinonide ointment (HALOG® ointment [Halcinonide Ointment, USP, 0.1%) for treating moderate to severe plaque psoriasis. The study protocol was approved by an institutional review board and the study was conducted in accordance with ethical guidelines. Written informed consent was obtained by all patients before initiating treatment.

Adults (≥18 years) with chronic, moderate to severe, plaque-type psoriasis (BSA≥10%, physician's global assessment [PGA]≥3, and psoriasis area severity index [PASI]≥12) who were candidates for phototherapy and/or systemic therapy were recruited. Exclusion criteria included active non-plaque forms of psoriasis; lab abnormality or medical conditions that could affect patient safety during the study; active or untreated latent tuberculosis; prior or concurrent malignancy; hepatitis B; recent treatment of psoriasis with UVB or PUVA phototherapy, oral systemic medications, biologics, or topical therapies; recent use of antibiotics or any investigational drug; pregnancy or breast feeding; and hypersensitivity to the excipients of study drugs. Patients who have received live vaccine within 4-weeks prior to baseline or intend to receive live vaccine during the study were also excluded.

### Study Treatment

All enrolled patients received tildrakizumab (100 mg) by subcutaneous injection at weeks 0, 4, and 16. Patients with BSA>3% at week 16 applied halcinonide ointment twice daily for 4 weeks and then continued without halcinonide for an additional 4 weeks of follow up. Patients with BSA≤3% at week 16 were also followed up to week 24. All patients were evaluated for safety and efficacy at weeks 4, 8, 16, 20 and 24.

### Study Outcomes

The primary endpoint of the study was the proportion of patients with BSA≤3% at week 16. Secondary endpoints included proportions of patients with BSA≤1%, dermatology life quality index (DLQI) of 0 or 1, and reduction of PASI score from baseline by 75%, 90%, and 100% (PASI 75, PASI 90, and PASI 100, respectively) at weeks 16, 20, and 24, in addition to proportions of patients with BSA≤3% at weeks 20 and 24. Improvements in PGA, the composite PGA×BSA measure, and DLQI were also evaluated as secondary endpoints. Safety outcomes included adverse events (AEs) and serious AEs (SAEs).

### Statistical Analysis

A cohort of approximately 25 participants was planned for enrollment in the study. Changes from baseline in BSA, PGA, PGA×BSA, PASI, and DLQI were summarized descriptively at weeks 4, 8, 16, 20, and 24; no formal statistical analyses were conducted given the sample size. AEs and SAEs were summarized descriptively by frequency and severity, and their causal relationship to treatment was assessed.

### Results

### Patient Disposition and Demographics

A total of 25 patients were enrolled and 19 completed the study; 1 discontinued the study due to no response to treatment, and 5 were lost to follow up.

The majority of the patients were male (68%) and white (76%), with a mean age of 52.6 years. Patients had psoriasis for an average of 18.9 years. Mean baseline BSA was 19.1%, and mean baseline scores were 3.5 for PGA, 16.7 for PASI, and 16.5 for DLQI. At week 16, 12 patients had BSA≤3%, while 11 had BSA>3% and received additional halcinonide ointment for 4 weeks.

### BSA and PGA Responses

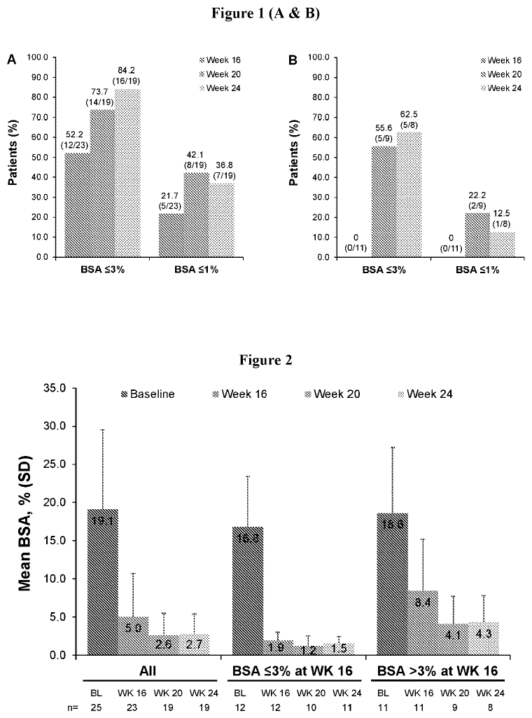

The proportion of all patients having affected BSA≤3% with tildrakizumab was 52.2% at week 16. This percentage of all patients (both ≤3% and >3% BSA at week 16) increased to 73.7% at week 20 (after 4 weeks of additional halcinonide ointment applied in those with an unsatisfactory, week-16 response), and reached 84.2% at week 24 after patients had not been using halcinonide ointment for 4 weeks (FIG. 1A). In the patients who added halcinonide therapy at week 16, 4 achieved BSA≤3% at week 20 and maintained the response level through week 24, 1 achieved BSA≤3% at week 20 but lost the response at week 24 (BSA 6%), and 1 achieved BSA≤3% at week 24, resulting in total 62.5% of this group achieving BSA≤3% at study end (FIG. 1B). Most patients who had BSA≤3% at week 16 (no halcinonide use) and completed the study maintained a BSA≤3% at weeks 20 and 24; the BSA of 1 patient increased to 4% at week 20, but dropped back to 3% at week 24. The proportion of all patients with BSA≤1% also increased from 21.7% at week 16 to 42.1% at week 20, and was 36.8% at week 24 (FIG. 1A). In patients who used halcinonide, 22.2% attained BSA≤1% at week 20 and 12.5% at week 24 (FIG. 1B).

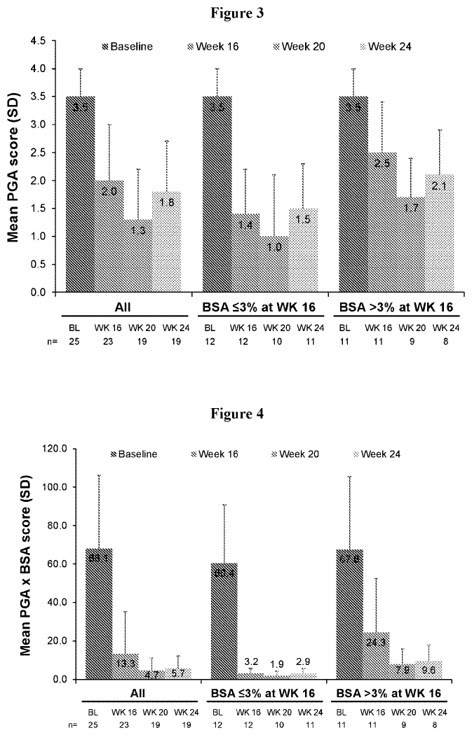

Mean BSA involvement for all patients decreased from 19.1% at baseline to 5.0% at week 16 (74% reduction), 2.6% at week 20, and 2.7% at week 24 (86% reduction for both) (FIG. 2). Mean PGA and PGA×BSA of all patients also improved with treatment. In patients who had BSA >3% at week 16, the reduction from baseline in mean BSA, PGA, and PGA×BSA was 55%, 29%, and 64%, respectively, at week 16, and was 78%, 51%, and 88%, respectively, at week 20 after 4 weeks of adjunctive halcinonide therapy; these responses were maintained at week 24 after halcinonide ointment had been stopped for 4 weeks. In patients who had BSA≤3% at week 16 and did not use halcinonide, the mean scores of BSA, PGA, and PGA×BSA were reduced by 89%, 60%, and 95%, respectively, from baseline to week 16, and the responses were maintained through week 24 (FIG. 2-4).

### PASI Responses

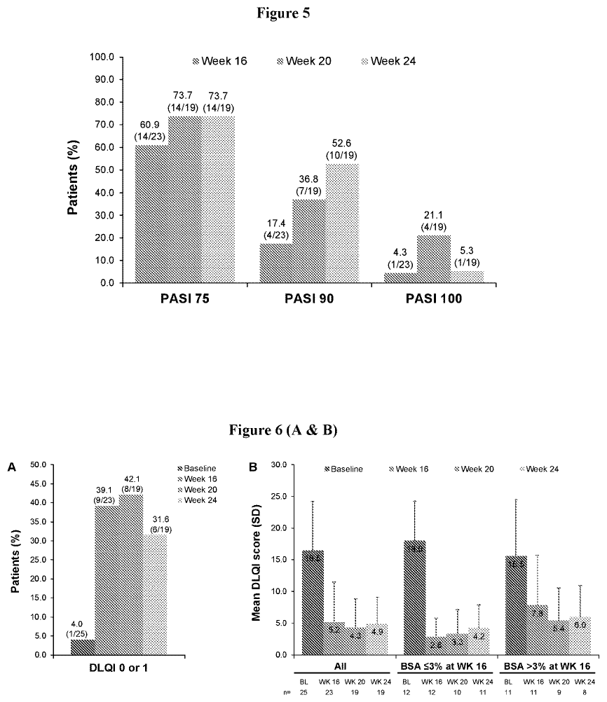

PASI 75 (≥75% reduction in PASI score versus baseline) was attained by 60.9% of all patients at week 16 in response to tildrakizumab monotherapy, and the percentage increased to 73.7% at weeks 20 and 24. The proportion of all patients achieving PASI 90 also increased from 17.4% at week 16 to 52.6% at week 24. PASI 100 (complete resolution) was achieved in 4.3% of all patients at week 16 and 21.1% at week 20, although the percentage dropped to 5.3% 4 weeks after halcinonide therapy was stopped (FIG. 5).

### Quality of Life

With 16 weeks of tildrakizumab monotherapy, the proportion of all patients reporting a DLQI score of 0 or 1 increased from 4.0% at baseline to 39.2% at week 16, and continued to increase at week 20 (42.1%), but decreased to 31.6% at week 24 (FIG. 6A).

Mean DLQI of all patients decreased from baseline to week 16 by 68% and the improvement was maintained through week 24. In patients who had BSA>3% and used additional halcinonide ointment, the reduction in mean DLQI from baseline increased from 50% at week 16 to 65% at week 20 and 61% at week 24. In patients who had BSA≤3% at week 16 and did not use halcinonide ointment, mean DLQI improved from baseline to week 16 by 84% and remained at similar levels at weeks 20 and 24 (FIG. 6B).

1. A method for treating a patient suffering from moderate to severe plaque psoriasis, which method comprises:
selecting a patient who has
a PASI score of ≥12
≥10% body surface area (BSA) affected by psoriasis and/or
a PGA score of ≥3
administering an IL-23 inhibitor to said patient at a dose of about 100 milligrams by subcutaneous injection at weeks 0, 4 and 16 week;
at week 16, applying a corticosteroid twice daily to the patients with BSA >3% for 4 weeks and then continued without the corticosteroid for an additional four weeks follow up. and
changes from baseline in BSA, PGA, PGA×BSA, PSAI and DLQI are noted; wherein the patients receiving the corticosteroid achieve BSA≤1% to ≤3% at week 20 and maintain the response level through week 24.

2. The method according to claim 1, wherein the percentage of reduction from baseline in mean BSA, PGA, and PGA×BSA significantly increases at week 20 after 4 weeks of adjunctive corticosteroid therapy and the response is maintained at week 24 after corticosteroid therapy stops for 4 weeks.

3. The method according to claim 1, wherein the percentage of reduction in PSAI (75, 90 and 100) from baseline significantly increases at week 20 after 4 weeks of adjunctive corticosteroid therapy and the response is maintained at week 24 after corticosteroid therapy stops for 4 weeks.

4. The method according to claim 1, wherein the reduction in DLQI from baseline significantly increases at week 20 after 4 weeks of adjunctive corticosteroid therapy and the response is maintained at week 24 after corticosteroid therapy stops for 4 weeks.

5. The method according to claim 1, wherein the corticosteroid is administered as a topical preparation.

6. The method according to claim 5, wherein the topical preparation is in the form of an ointment, a powder, a gel ointment, an emollient, a cream, an adhesive patch or strip, a shampoo, a spray, foam, a gel, a liquid or a solution.

7. The method according to claim 5. wherein the corticosteroid is administered as an ointment.

8. The method according to claim 1, wherein the corticosteroid is selected from beclomethasone dipropionate, betamethasone valerate, budesonide, clobetasol propionate, clobetasone butyrate, clobetasol propionate, clocortolone acetate, clocortolone pivalate, corticosterone, cortisone, esoximetasone acetatefluocinolone acetonide, halcinonide, halobetasol propionate, hydrocortisone acetate, hydrocortisone butyrate, hydrocortisone probutate, hydrocortisone phosphate, hydrocortisone succinate sodium, methylprednisolone acetate, prednisolone, and prednisone.

9. The method according to claim 1. wherein the IL-23 inhibitor is selected from guselkumab. risankizumab, and tildrakizumab.

10. The method according to claim 1. wherein the IL-23 antagonist is tildrakizumab.

11. A method for treating a psoriasis patient who is receiving an IL-23 antagonist comprising continuing treatment with the IL-23 antagonist, initiating treatment with a topical preparation containing a corticosteroid, continuing treatment with the topical preparation containing the corticosteroid for not more than 4 weeks and then discontinuing treatment with the topical preparation, wherein the patient percentage achieving BSA≤3% is increased after just 4 weeks of additional corticosteroid applied to those who did not achieve an adequate response to IL-23 antagonist alone, and the said improvements are maintained 4 weeks after corticosteroid treatment is stopped.

12. The method according to claim 11, wherein the percentage of reduction from baseline in mean BSA, PGA, and PGA×BSA significantly increases at week 20 after 4 weeks of adjunctive corticosteroid therapy; the percentage of reduction in PSAI (75, 90 and 100) from baseline significantly increases at week 20 after 4 weeks of adjunctive corticosteroid therapy; the reduction in DLQI from baseline significantly increases at week 20 after 4 weeks of adjunctive corticosteroid therapy; and the response is maintained at week 24 after corticosteroid therapy stops for 4 weeks.

13. The method accosting to claim 11, wherein the patient continues the treatment with the IL-23 antagonist for at least about 16 weeks.

14. The method according to claim 11, wherein the IL-23 antagonist is guselkumab, risankizumab or tildrakizumab.

15. The method according to claim 11, wherein the IL-23 antagonist is tildrakizumab.

16. The method according claim 11, wherein the corticosteroid is administered as a topical preparation.

17. The method according to claim 16, wherein the topical preparation is in the form of an ointment, a powder, a gel ointment, an emollient, a cream, an adhesive patch or strip, a shampoo, a spray, foam, a gel, a liquid or a solution.

18. The method according to claim 11, wherein the corticosteroid is selected from beclomethasone dipropionate, betamethasone valerate, budesonide, clobetasol propionate, clobetasone butyrate, clobetasol propionate, clocortolone acetate, clocortolone pivalate, corticosterone, cortisone, esoximetasone acetatefluocinolone acetonide, halcinonide, halobetasol propionate, hydrocortisone acetate, hydrocortisone butyrate, hydrocortisone probutate, hydrocortisone phosphate, hydrocortisone succinate sodium, methylprednisolone acetate, prednisolone, prednisone, and halcinonide.

19. The method according to claim 16, wherein the corticosteroid is a halcinonide ointment.
